(19) United States
(12) Patent Application Publication
(10) Pub. No.: US 2019/0136167 A1
(43) Pub. Date: May 9, 2019

(54) SYSTEMS AND METHODS FOR CREATING A MIXED COCKTAIL DRINK

(71) Applicants: Reid Rosenthal, Philadelphia, PA (US); Aaron Grant Kaplowitz, Philadelphia, PA (US); Arielle Katz Kaplowitz, Philadelphia, PA (US)

(72) Inventors: Reid Rosenthal, Philadelphia, PA (US); Aaron Grant Kaplowitz, Philadelphia, PA (US); Arielle Katz Kaplowitz, Philadelphia, PA (US)

(21) Appl. No.: 15/806,941

(22) Filed: Nov. 8, 2017

(51) Int. Cl. C12G 3/06; A47J 43/27; A61K 31/05; A61K 31/352; A61K 9/00; B65D 43/02; B65D 85/72

(52) U.S. Cl. CPC C12G 3/06; A47J 43/27; A61K 31/05; B65D 85/72; A61K 9/0095; B65D 43/02; A61K 31/352

(57) ABSTRACT

A flavored cube can be used to provide ingredients for mixing a cocktail and to keep the cocktail chilled without diluting the taste or potency of the cocktail. The flavored cube can be packaged in a variety of ways, including individually packaged units and/or kits that include a plurality of flavored cubes.

### CROSS-REFERENCE TO RELATED APPLICATIONS

This application claims the benefit of and is a continuation of U.S. Non-Provisional patent application Ser. No. 14/789,956; filed Jul. 1, 2015; and entitled SYSTEMS AND METHODS FOR CREATING A MIXED COCKTAIL DRINK. The entire contents of U.S. patent application Ser. No. 14/789,956 are incorporated by reference herein. U.S. Non-Provisional patent application Ser. No. 14/789,956 claims the benefit of U.S. Provisional Patent Application No. 62/021,295, filed Jul. 7, 2014; and entitled SYSTEMS AND METHODS FOR CREATING A MIXED COCKTAIL DRINK. The entire contents of U.S. Patent Application No. 62/021,295 are incorporated by reference herein.

### BACKGROUND

### Field

Various embodiments disclosed herein relate to devices and methods for mixing a cocktail drink.

### Description of Related Art

A cocktail is a mixed drink that is typically made with a distilled alcohol (such as gin, brandy, vodka, whiskey, tequila, or rum) and mixed with other ingredients. The ingredients can include one or more types of juice, fruit, sauce, honey, milk, cream, spices, or any other flavorings. Consequently, some cocktails can require numerous ingredients and tedious mixing procedures.

Additionally, conventional cocktails often include ice cubes to help keep the cocktail chilled. As the ice cubes melt, this can often dilute the taste and potency of the cocktail. A system and method is needed to reduce the number of ingredients, ease the mixing process, as well as keep the cocktail chilled without diluting the taste and potency.

### SUMMARY

Several embodiments can include a method for mixing a cocktail drink. The method can include placing a flavored frozen cube into a beverage container; pouring a predetermined amount of alcohol into the beverage container; and causing at least a portion of the flavored frozen cube to melt.

The flavored frozen cube can be enclosed in a packaging container. Methods can further include removing a lid member that is detachably coupled to the packaging container; removing the flavored frozen cube from the packaging container; and pouring the predetermined amount of alcohol into the packaging container to thereby measure the amount of alcohol.

In some embodiments, the flavored frozen cube can define a flavor profile selected from the group consisting of: 1) a first flavor profile consisting of cucumber juice, watermelon puree and thyme; 2) a second flavor profile consisting of pink grapefruit juice, chili and tarragon; 3) a third flavor profile consisting of blood orange, simple syrup, cherry juice, bitters and ginger; 4) a fourth flavor profile consisting of Meyer lemon juice, bergamot, vanilla essence and chamomile; and 5) a fifth flavor profile consisting of peach nectar, Meyer lemon juice and Benedictine herbs.

As well, in some embodiments, the predetermined amount of alcohol is at least 1.4 ounces. Even still, in some embodiments, the flavored frozen cube can comprise cannabidiol (CBD). Furthermore, in some embodiments, the flavored frozen cube can comprise tetrahydrocannabinol (THC).

The beverage container can be a martini shaker. Accordingly, methods can further include coupling a cover to an open end of the martini shaker; and shaking the martini shaker until the flavored frozen cube has melted.

The disclosure also includes a flavored cube for mixing a cocktail drink. The flavored cube can include a packaging container; a lid member detachably coupled to the packaging container; and the flavored cube located inside the packaging container, the flavored cube having a liquid state and a frozen state.

In some embodiments, the flavored cube can comprise cannabidiol (CBD). As well, in some embodiments, the flavored cube can comprise tetrahydrocannabinol (THC).

The packaging container can define an internal space substantially equal to 30 milliliters. In the liquid state, the flavored cube can define a volume substantially equal to 25 milliliters. In the frozen state, the flavored cube can define a volume of less than 30 millimeters.

The packaging container can comprise plastic. The lid member can be adhesively attached to the packaging container. As well, the flavored cube can comprise at least three flavors.

In some embodiments, the at least three flavors can define a flavor profile selected from the group consisting of: 1) a first flavor profile consisting of cucumber juice, watermelon puree and thyme; 2) a second flavor profile consisting of pink grapefruit juice, chili and tarragon; 3) a third flavor profile consisting of blood orange, simple syrup, cherry juice, bitters and ginger; 4) a fourth flavor profile consisting of Meyer lemon juice, bergamot, vanilla essence and chamomile; and 5) a fifth flavor profile consisting of peach nectar, Meyer lemon juice and Benedictine herbs.

The disclosure also includes a kit for mixing a cocktail drink. The kit can include a first packaging container and a second packaging container each defining an internal space to receive a first flavored cube and a second flavored cube, respectively; the first flavored cube comprising a first plurality of ingredients, the first flavored cube having a liquid state and a frozen state; the second flavored cube comprising a second plurality of ingredients, the second flavored cube having a liquid state and a frozen state; and a first lid member and a second lid member that are each detachably coupled to the first packaging container and the second packaging container, respectively, wherein the first and second lids substantially seal the first and second packaging containers to thereby prevent the flavored cubes from spilling from the first and second packaging containers.

At least one of the first plurality of ingredients and the second plurality of ingredients can comprise cannabidiol (CBD). As well, at least one of the first plurality of ingredients and the second plurality of ingredients can comprise tetrahydrocannabinol (THC).

In some embodiments, the kit can further include a holding tray that receives the first and second packaging containers. The holding tray can be configured to receive at least 12 packaging containers.

As well, in some embodiments, the kit can further include at least one of a martini shaker and a lowball glass. Even still, in some embodiments, at least one of the first plurality of ingredients and the second plurality of ingredients can define a flavor profile selected from the group consisting of: 1) a first flavor profile consisting of cucumber juice, watermelon puree and thyme; 2) a second flavor profile consisting of pink grapefruit juice, chili and tarragon; 3) a third flavor profile consisting of blood orange, simple syrup, cherry juice, bitters and ginger; 4) a fourth flavor profile consisting of Meyer lemon juice, bergamot, vanilla essence and chamomile; and 5) a fifth flavor profile consisting of peach nectar, Meyer lemon juice and Benedictine herbs. Furthermore, in some embodiments, the internal space of the first and second packaging containers can each define a volume substantially equal to 30 milliliters.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments.

FIGS. 1 and 2 illustrate a process for mixing a cocktail.

FIG. 3 illustrates an embodiment of a packaging container for a flavored cube.

FIG. 4 illustrates a process of removing a detachable lid from the packaging container.

FIGS. 5, 6, 7 and 8 illustrate another process for mixing a cocktail.

FIGS. 9 and 10 illustrate a system for storing and dispensing a plurality of flavored frozen cubes.

FIG. 11 illustrates a kit container for holding a plurality of flavored cubes.

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Many people consume alcoholic and nonalcoholic mixed drinks in their homes and other establishments, such as bars and nightclubs. Oftentimes these drinks are mixed using ingredients that may be difficult to access and even sometimes involve complex mixing procedures. One occupation that has gained popularity in recent years is that of the mixologist. A mixologist can refer to a person who studies and helps evolve the field of bartending, by creating innovative cocktails and refining the techniques and drinks of old. Mixologists can also be cocktail historians that seek to introduce and revive cocktails from previous generations.

However, unless a person possesses the fine training of a mixologist and has access to all the required ingredients, creating some mixed drinks can be difficult and/or costly. As such, this disclosure includes several embodiments that can reduce the complexities involved in mixing finely crafted cocktails, thus, making some cocktails more widely available. It should be appreciated that the words cocktail, cocktail drink, mixed drink, beverage, martini, and the like can be used interchangeably throughout this disclosure.

### Flavored Cube Embodiments

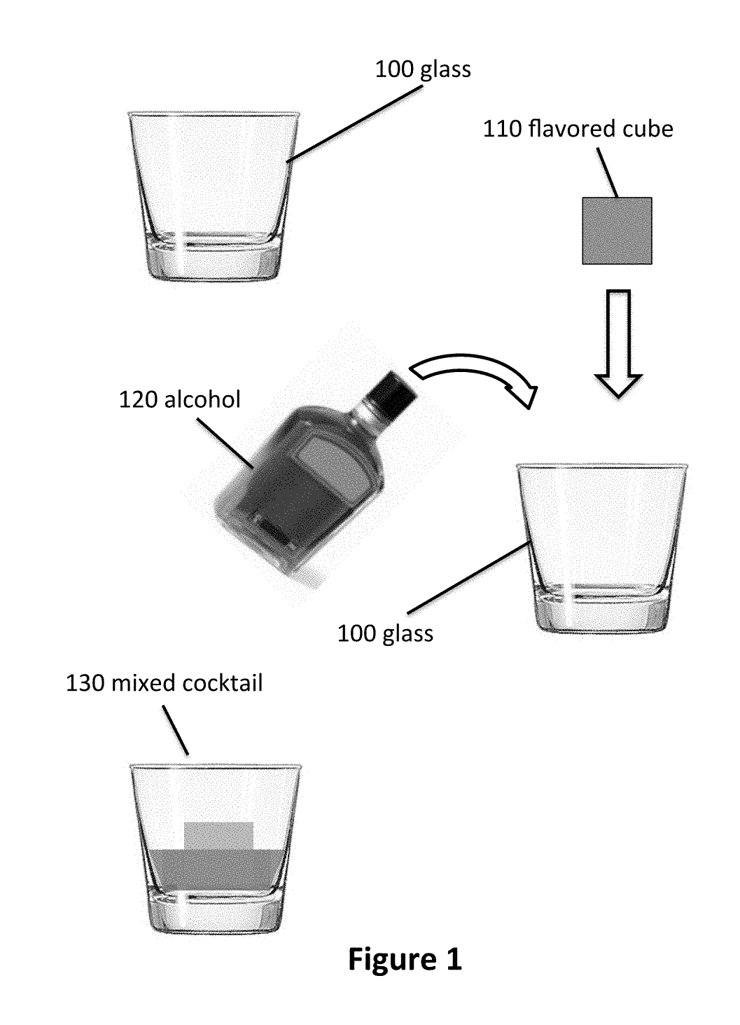

FIG. 1 illustrates a method for mixing a cocktail drink. The method can include placing a flavored cube 110 into a glass 100, or any type of beverage container. The flavored cube 110 can have a liquid state and a frozen state. As such, it should be appreciated that the flavored cube 110, in the frozen state, can be referred to as a flavored frozen cube, while the flavored cube 110, in the liquid state, can be referred to as a flavored liquid cube.

Some embodiments can include pouring a predetermined amount of alcohol 120 into the glass 100 to produce a mixed cocktail 130. However, it should also be appreciated this disclosure can include embodiments that are nonalcoholic. As such, some methods can include pouring a predetermined amount of liquid into the glass 100 to produce a mixed beverage. For example, the liquid can be water, or any other nonalcoholic liquid.

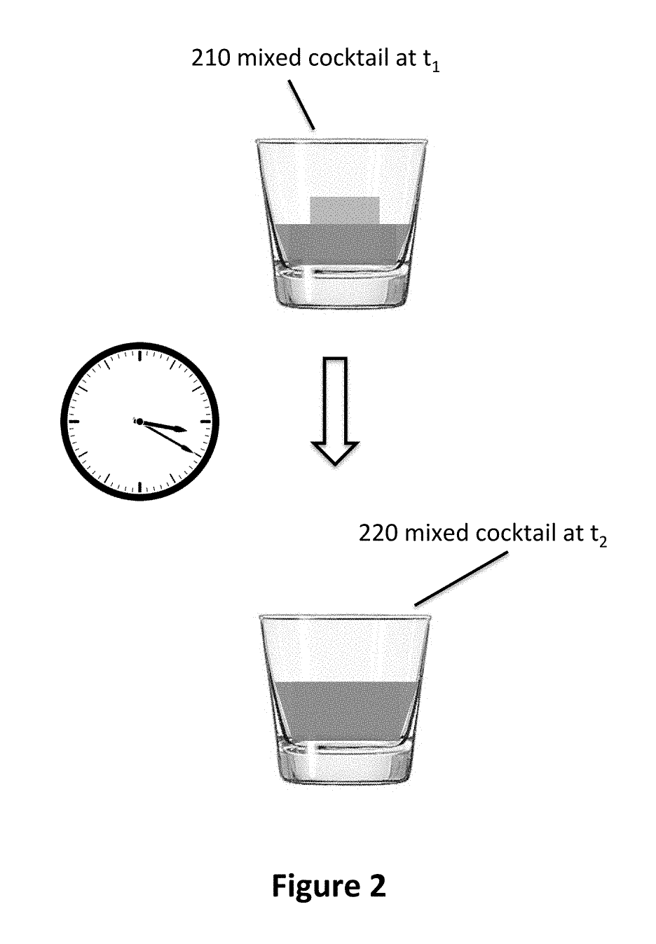

The method can include causing at least a portion of the flavored cube 110 to melt, to thus create a mixed drink, such as a cocktail. The flavored cube 110 can be caused to melt for a variety of reasons. In the embodiment illustrated in FIG. 2, the mixed cocktail can be placed in an ambient temperature that is warmer than the flavored cube 110 and, as such, the mere passage of time in this environment can cause the flavored cube to melt. Specifically, the melting of the flavored cube 110 can be shown by the illustration of the mixed cocktail at time t₁ 210, which shows the flavored cube 110 substantially unmelted, and the mixed cocktail at time t₂ 220, which shows the flavored cube 110 substantially melted. It should be appreciated that time t₁ can be any time before time t₂.

However, the flavored cube 110 can be caused to melt for a variety of reasons. In some embodiments, the flavored cube 110 can be caused to melt by contact with the alcohol 120 or nonalcoholic liquid, stirring of the liquid or alcohol in which the flavored cube 110 is submersed, or any other type of action that would cause the flavored cube 110 to melt. In some embodiments, the method can further include mixing the alcohol 120 or liquid with the flavored cube 110 until at least 50% of the flavored cube 110 has melted. Yet some methods can include mixing the alcohol 120 or liquid with the flavored cube 110 until all of the flavored cube 110 has melted. However, it should be appreciated that any amount of the flavored cube 110 can be caused to melt.

The flavored cube 110 can include a plurality of ingredients, which can further define a flavor profile. The flavor profile may consist of or comprise any number of ingredients. Specifically, when Applicant claims a flavor profile that comprises, this should be understood to mean that the flavor profile includes at least the stated flavors and potentially other flavors as well. However, when the Applicant claims a flavor profile that consists of this should be understood to mean that the flavor profile consists of the stated flavors and includes no additional flavors.

For example, in one embodiment, the flavor profile can be selected from the group consisting of: 1) a first flavor profile that consists of cucumber juice, watermelon puree and thyme; 2) a second flavor profile that consists of pink grapefruit juice, chili and tarragon; 3) a third flavor profile that consists of blood orange, simple syrup, cherry juice, bitters and ginger; 4) a fourth flavor profile that consists of Meyer lemon juice, bergamot, vanilla essence and chamomile; and 5) a fifth flavor profile that consists of peach nectar, Meyer lemon juice and Benedictine herbs.

However, it should also be appreciated that some embodiments may comprise, rather than consist of any of the previously stated ingredients. For example, the flavor profile can be selected from the group consisting of: 1) a first flavor profile that comprises cucumber juice, watermelon puree and thyme; 2) a second flavor profile that comprises pink grapefruit juice, chili and tarragon; 3) a third flavor profile that comprises blood orange, simple syrup, cherry juice, bitters and ginger; 4) a fourth flavor profile that comprises Meyer lemon juice, bergamot, vanilla essence and chamomile; and 5) a fifth flavor profile that comprises peach nectar, Meyer lemon juice and Benedictine herbs. Stated differently,

Furthermore, specific types of alcohol 120 or liquids can be coupled with specific combinations of ingredients. For example, in one embodiment, the alcohol 120 can be tequila and the plurality of ingredients can comprise cucumber juice, watermelon puree and thyme. In another embodiment, the alcohol 120 can be gin and the plurality of ingredients can comprise pink grapefruit juice, chili and tarragon. In yet another embodiment, the alcohol 120 can be bourbon and the plurality of ingredients can comprise blood orange, simple syrup, cherry juice, bitters and ginger. In another embodiment, the alcohol can be rum and the plurality of ingredients can comprise Meyer lemon juice, bergamot, vanilla essence and chamomile. Furthermore, in yet another embodiment, the alcohol can be vodka and the plurality of ingredients can comprise peach nectar, Meyer lemon juice and Benedictine herbs. While the examples described above pair specific alcohols with certain ingredients, it should be appreciated that in some embodiments the alcohol 120 can be paired with any of the ingredients. Generally, the alcohol can be selected from the group consisting of vodka, bourbon, whiskey, rum, gin, tequila, champagne, beer, wine and any other type of alcohol used to make mixed cocktails. As well, in some embodiments, a nonalcoholic liquid can be mixed with the flavored cube 110. For example, the nonalcoholic liquid can be selected from the group consisting of club soda, tonic water, seltzer and any other type of liquid used to mix a drink.

As well, in some embodiments, the plurality of ingredients comprises any type of cannabis (or marijuana). It should be appreciated that with the recent legislation to legalize cannabis in certain jurisdictions, such as Colorado, that inclusion of cannabis as an ingredient is now patentable subject matter. Specifically, in some embodiments, the plurality of ingredients can include cannabidiol (CBD) or tetrahydrocannabinol (THC). However, in some embodiments, the plurality of ingredients can include both cannabidiol (CBD) and tetrahydrocannabinol (THC). It should also be appreciated that the plurality of ingredients of the flavored cubes 110 may comprise any other legalized drug, either on its own, or in combination with any other legalized drug.

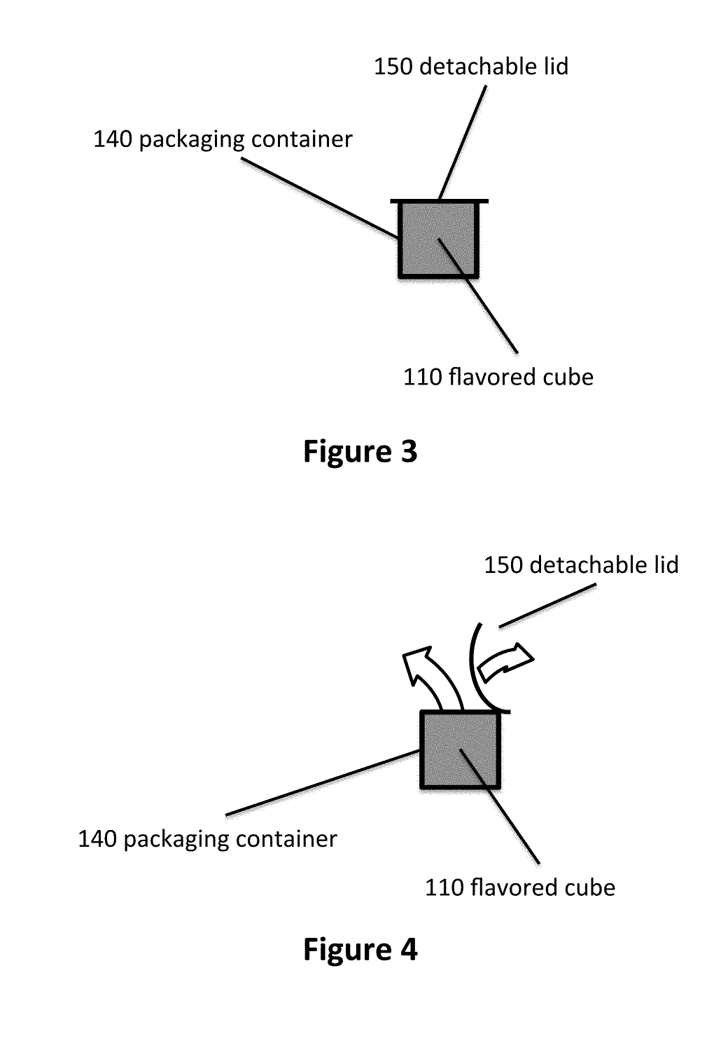

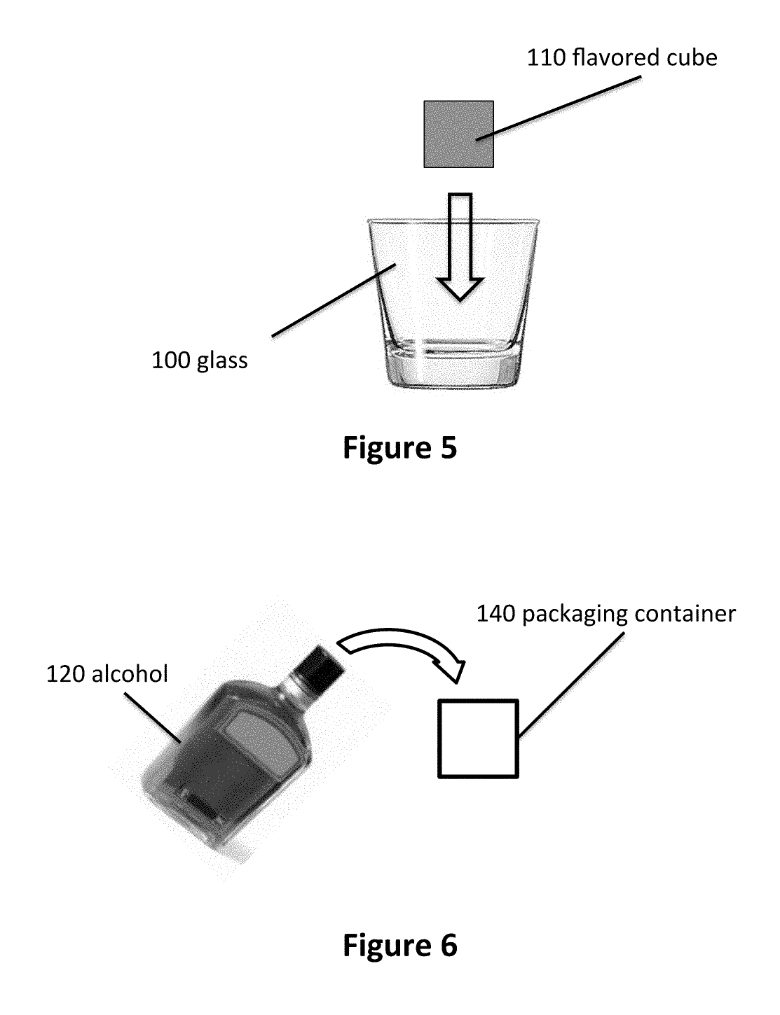

As illustrated in FIGS. 3 and 4, in some embodiments, the flavored cube 110 is contained in a packaging container 140 that is enclosed by a detachable lid 150. The detachable lid 150 can be configured to seal the internal space of the packaging container 140 to thereby prevent the contents of the flavored cube 110 from spilling from the packaging container 140. As well, the detachable lid 150 can be configured to be detachable from the packaging container 140 so as to allow the flavored cube 110 to be removed from the packaging container 140. Accordingly, and as shown in FIG. 4, some embodiments can further include removing the flavored cube 110 from the packaging container 140. As illustrated in FIG. 5, the flavored cube 110 can be placed in the glass 100.

With continued reference to the packaging container 140, the packaging container 140 can define an internal space that is substantially equal to 30 milliliters. In the liquid state, the flavored cube 110 can define a volume that is substantially equal to 25 milliliters. In the frozen state, the flavored cube 110 can define a volume that is less than 30 millimeters. However, it should be appreciated that the volume of the internal space can define any size. As well, the volume of the flavored cube 110, in either the liquid state or the frozen state, can define any size.

The packaging container 140 can be constructed of plastic, or any suitable material that is able to withstand a wide range of temperatures (such as from −40° F. to 250° F.). As well, the detachable lid 150 can be adhesively attached to the packaging container. However, it should be appreciated that the detachable lid 150 can be attached to the packaging container 140 via any detachable connection.

Furthermore, the method can include measuring a predetermined amount of alcohol 120. As illustrated in FIG. 6, the method can include pouring the predetermined amount of alcohol into the packaging container 140 to thereby measure the amount of alcohol. However, it should be appreciated that the alcohol 120 can be measured using any type of container suitable for holding liquid, such as a shot glass, measuring cup, and the like. It should also be appreciated that the predetermined amount of alcohol can be any amount of alcohol, for example, the predetermined amount of alcohol can be at least 1.4 ounces.

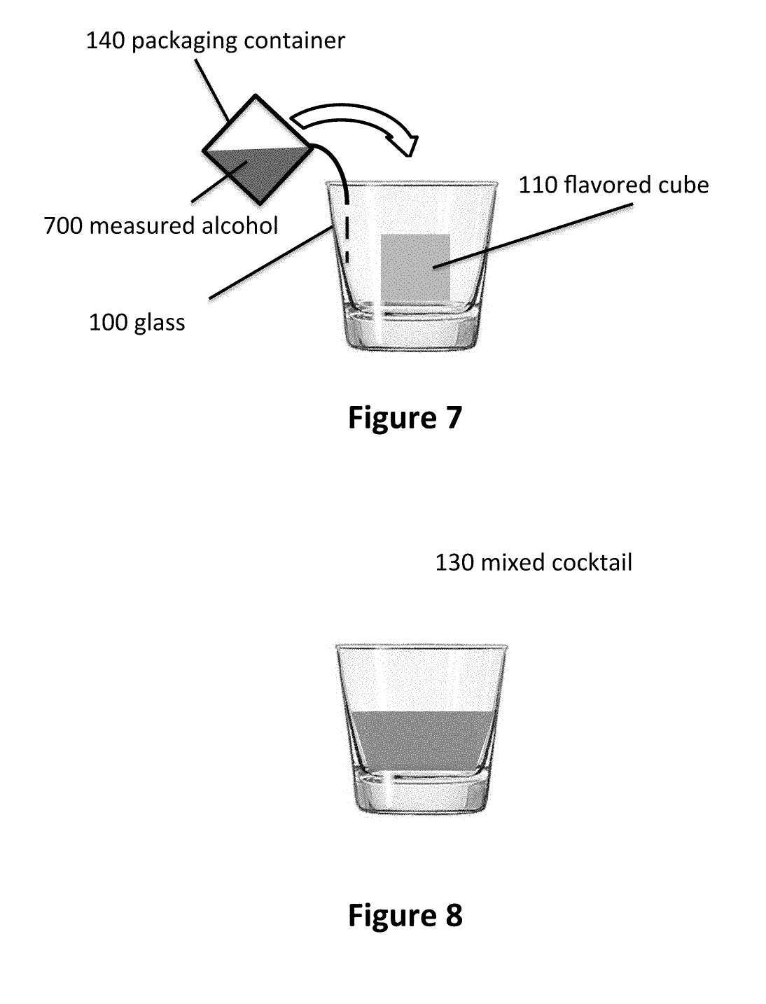

As illustrated in FIG. 7, the method can include pouring the predetermined amount of alcohol, or measured alcohol 700, into the glass 100, such that the alcohol 120 thereby contacts the flavored cube 110. As illustrated in FIG. 8, and as previously discussed with reference to FIG. 2, after the flavored cube 110 melts, the resulting mixture of the alcohol 120 and the flavored cube 110 can be the mixed cocktail 130.

In some embodiments, the beverage container can be a martini shaker. In this manner, the method can further include coupling a martini shaker top to a open end of the martini shaker. As well, the method can also include shaking the martini shaker until at least 50% of the flavored cube 110 has melted. Even still, the method can further include shaking the martini shaker until the entire flavored cube 110 has melted.

### Bottle Service Embodiments

Some establishments that serve beverages, such as bars and nightclubs, offer a service known as bottle service. Bottle service typically includes a reserved table for the customer's party, as well as bottles of alcohol 120, as selected by the customer, and mixers of the customer's choice. Bottle service can also include the service of a VIP host, or member of the wait staff, who can ensure that customers have sufficient mixers and can often make drinks using the alcohol 120 and mixers provided.

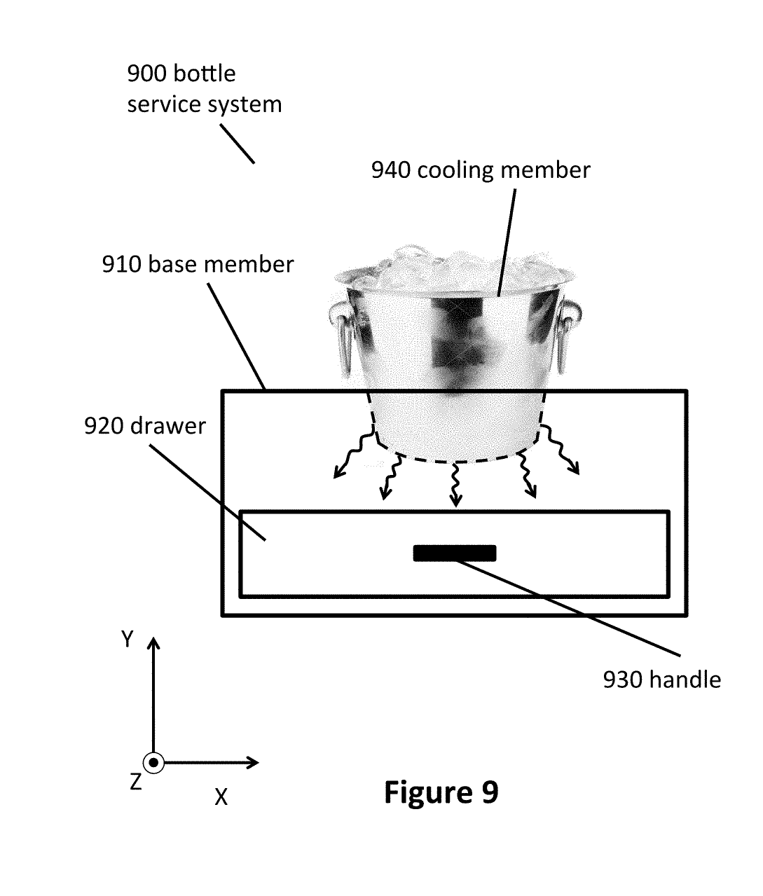

As such, some embodiments can include a method for providing a cocktail drink to a bottle service customer. As well, the method can be performed by any employee of the nightclub or bar, such as a cocktail server, bartender, VIP host, and the like. The method can include providing, to the customer, a bottle service system 900 for storing and dispensing a plurality of flavored cubes 110, like the one illustrated in FIG. 9. As well, the method can include receiving instructions from the customer to place a flavored cube 110 comprising a plurality of ingredients into a beverage container, such as a glass 100.

Bottle service customers can be seated on couches or chairs that are near adjacent tables for setting drinks, food, and the like. Accordingly, the method can also include placing the bottle service system 900 for storing and dispensing the plurality of flavored frozen cubes on a tabletop. However, it should be appreciated that the bottle service system 900 can be placed on a bar top, a floor, a shelf, or any place that is accessible to a customer. For example, in some embodiments the bottle service system 900 can be mounted on a wall or directly to the chair or couch that the customer is seated.

Some methods can further include removing the detachable lid 150 and/or removing the flavored cube 110 from the packaging container 140. Some methods can also include placing the flavored cube 110 into the beverage container. Accordingly, some methods can include measuring a predetermined amount of alcohol in the packaging container 140 and pouring the predetermined amount of alcohol 120 into the beverage container. The predetermined amount of alcohol can be any amount of alcohol. For example, in some embodiments, the predetermined amount of alcohol is at least 1.4 ounces. It should be appreciated that the alcohol can be any type of alcohol discussed in this disclosure.

Finally, the method can include causing at least a portion of the flavored cube 110 to melt. In some embodiments, the method includes mixing the alcohol 120 with the flavored cube 110 until at least 50% of the flavored cube 110 has melted. However, it should be appreciated that any amount of the flavored cube 110 can be melted.

In some embodiments the beverage container can be a martini shaker. In this manner, the method can further comprise coupling a martini shaker top to the open end of the martini shaker. Accordingly, the method can include shaking the martini shaker until the entire flavored cube 110 has melted. However, it should be appreciated that in some methods the martini shaker can be shaken until any amount of the flavored cube 110 has melted.

It should be appreciated that any of the steps disclosed herein can be performed by any employee of the establishment providing bottle service, the customer or anyone who has implicit consent from the customer.

While this disclosure may specifically refer to bottle service in nightclubs and/or bars, it should be appreciated this system can be used in any establishment that serves beverages. In some embodiments the system 900 can be used in a hotel or a facility that hosts parties or other events. For example, a hotel patron may order the system 900 to his or her room for a party. In this example, the hotel patron, a hotel employee, or anyone attending the patron's party may perform any of the method steps described above.

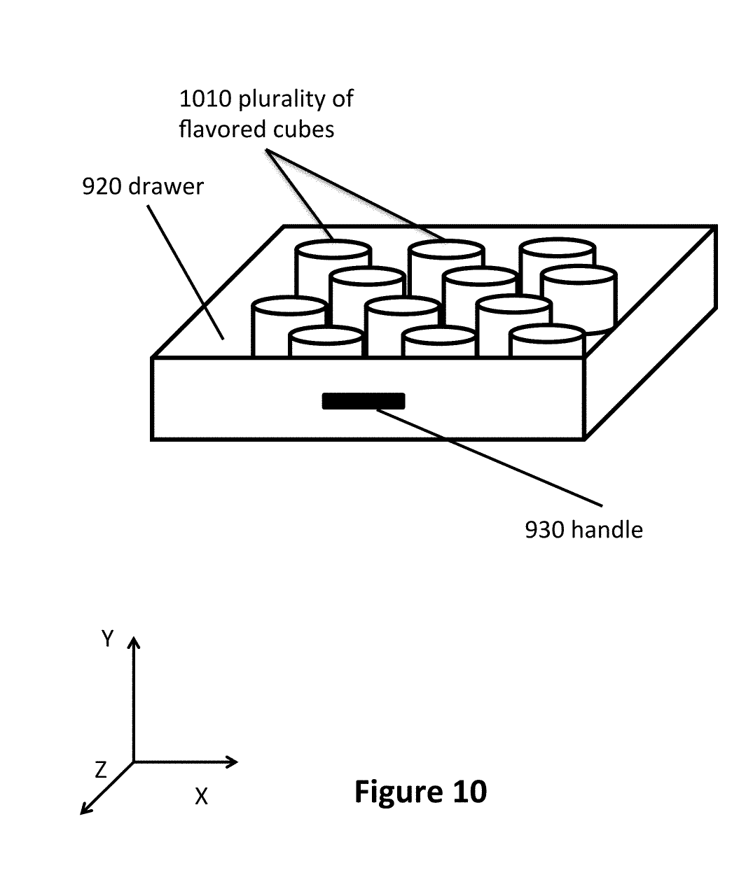

With specific reference to FIGS. 9 and 10, the bottle service system 900 for storing and dispensing a plurality of flavored frozen cubes 1010 can include a drawer 920 having a base surface that extends along a first direction (X) and sidewalls that protrude from an outer edge of the base surface and are elongate along a second direction (Y) that is perpendicular to the first direction (X). The drawer 920 can include a handle 930, and the drawer 920 can define an internal tray configured for storing and dispensing the plurality of flavored frozen cubes 1010. The bottle service system 900 can also include a base member 910 that can receive the drawer 920 within an internal space of the base member 910. Furthermore, the base member 910 can have: 1) a bottom surface that extends along the first direction (X), 2) a top surface that extends along the first direction (X) and is opposite the bottom surface, 3) sidewalls that extend along the second direction (Y) between the top and bottom surfaces, and 4) an internal space that is substantially enclosed between the sidewalls and the top and bottom surfaces. One of the sidewalls can define an opening that is configured to receive the drawer 920 into the internal space of the base member 910.

With continued reference to FIG. 9, the bottle service system 900 can also include a cooling member 940 that is detachably coupled to the top surface of the base member. The top surface can be constructed of a conductive material, such as aluminum. The top surface, via the cooling member 940, can be configured to keep the internal space, and thereby the contents of the drawer 920, chilled so as to prevent melting and maintain the frozen state of the plurality of flavored cubes 1010. It should be appreciated that the cooling member 940 can be any type of device configured to chill an environment. For example, and as illustrated in FIG. 9, the cooling member 940 can be an ice bucket, or a bowl for receiving a plurality of ice cubes. It should be appreciated that a plurality of ice cubes can be placed in the bowl. In another example, the cooling member 940 can be an ice block, a plurality of ice cubes, and the like.

The bottle service system 900 can include any type of accessory that is commonly used for bottle service. For example, some embodiments may include at least one bottle of alcohol placed in the bowl, or cooling member 940, such that the at least one bottle of alcohol is in physical contact with at least one ice cube of the plurality of ice cubes. As well, some embodiments can include at least one beverage container, such as the glass 100, that is detachably coupled to the base member. It should also be appreciated that the at least one beverage container can be detachably coupled to any part of the base member, for example the top surface of the base member or an exterior surface of at least one of the sidewalls of the base member.

### Kit Embodiments

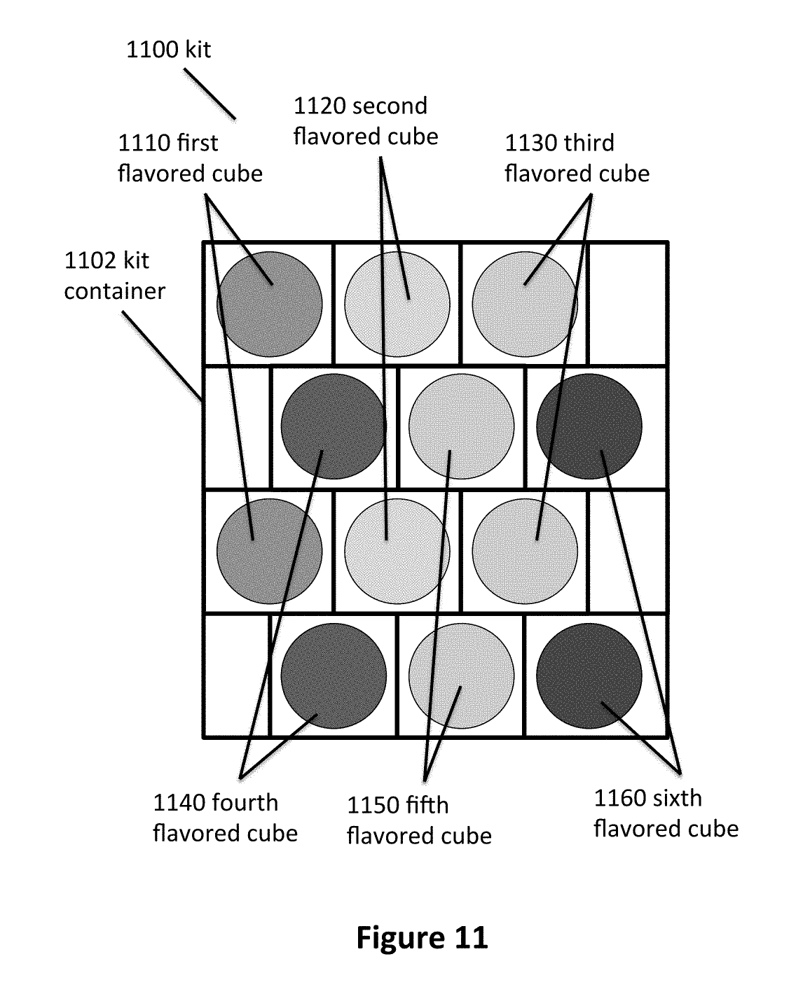

As illustrated in FIG. 11, the disclosure also includes a kit 1100 for mixing a cocktail drink. The kit 1100 can comprise a kit container 1102, or holding tray, that can include a first packaging container and a second packaging container; the first and second packaging containers can be the packaging container 140 described above. Each of the first and second packaging containers can define an internal space configured to receive a respective first flavored cube 1110 and a second flavored cube 1120, respectively. The first flavored cube 1110 can comprise a first plurality of ingredients, and the second flavored cube 1120 can comprise a second plurality of ingredients. It should be appreciated that the first flavored cube 1110 can be the same as the second flavored cube 1120. However, it should also be appreciated that the first flavored cube 1110 can be different from the second flavored cube 1120. For example, the first flavored cube 1110 can comprise cucumber juice, watermelon puree and thyme, while the second flavored cube 1120 can comprise pink grapefruit juice, chili and tarragon. Generally, it should be appreciated that any of the flavored cubes can comprise any of the ingredients previously disclosed. As such, any of the flavored cubes can include more than just the ingredients disclosed. However, any of the flavored cubes can be said to consist of any of the ingredients previously disclosed. As such, the flavored cubes can be limited to specific ingredients.

The kit container 1102 can be configured to receive any number of flavored cubes, each having a unique flavor that is the same or different from the other flavored cubes within the kit 1100. For example, and as illustrated in FIG. 11, some kit embodiments can include 12 total flavored cubes, with 6 unique flavors total, or 6 different flavored cubes (e.g. first flavored cube 1110, second flavored cube 1120, third flavored cube 1130, fourth flavored cube 1140, fifth flavored cube 1150, and sixth flavored cube 1160). However, it should be appreciated that the kit 1102 can be configured to include any number of flavored cubes that is less than 12, or greater than 12 flavored cubes. As well, any combination of first, second, third, fourth, fifth, and/or sixth flavored cubes can be included in the kit 1102.

As well, it should be appreciated that the kit container 1102 can be configured in any type of layout. For example, the rows and columns can be aligned, or offset as in FIG. 11. In yet other embodiments, the flavored cubes can be aligned in circular or triangular shapes. Yet in some other embodiments, the flavored cubes are not aligned in any pattern and are placed at random into the kit container 1102.

In some embodiments the kit 1100 can also include other accessories, such as the accessories previously discussed. For example, in some embodiments, the kit further comprises a beverage container. In some embodiments the beverage container can be a martini shaker. In yet other embodiments, the beverage container can be a lowball glass. The kit 1100 can also include other accessories, for example, a book that includes suggested food items to pair with any of the flavored cubes, such as the first flavored cube 1110 and/or the second flavored cube 1120.

### Interpretation

For the purposes of this disclosure the term “substantially” should be understood to mean for the most part or to a great or significant extent. For example, to say that a packaging container defines a volume that is substantially equal to 30 milliliters should be understood to mean that the volume is a number close to 30 millimeters, for example within ±0.5 millimeters.

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

Any section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section.

The various features and processes described above may be used independently of one another, or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

The following is claimed:

1. A method for mixing a cocktail drink, comprising:
placing a flavored frozen cube into a beverage container;
pouring a predetermined amount of alcohol into the beverage container; and
causing at least a portion of the flavored frozen cube to melt.

2. The method of claim 1, wherein the flavored frozen cube is enclosed in a packaging container, the method further comprising:
removing a lid member that is detachably coupled to the packaging container;
removing the flavored frozen cube from the packaging container; and
pouring the predetermined amount of alcohol into the packaging container to thereby measure the amount of alcohol.

3. The method of claim 2, wherein the flavored frozen cube define a flavor profile selected from the group consisting of: 1) a first flavor profile consisting of cucumber juice, watermelon puree and thyme; 2) a second flavor profile consisting of pink grapefruit juice, chili and tarragon; 3) a third flavor profile consisting of blood orange, simple syrup, cherry juice, bitters and ginger; 4) a fourth flavor profile consisting of Meyer lemon juice, bergamot, vanilla essence and chamomile; and 5) a fifth flavor profile consisting of peach nectar, Meyer lemon juice and Benedictine herbs.

4. The method of claim 2, wherein the predetermined amount of alcohol is at least 1.4 ounces.

5. The method of claim 1, wherein the flavored frozen cube comprises cannabidiol (CBD).

6. The method of claim 1, wherein the flavored frozen cube comprises tetrahydrocannabinol (THC).

7. The method of claim 1, wherein the beverage container is a martini shaker, the method further comprising:
coupling a cover to an open end of the martini shaker; and
shaking the martini shaker until the flavored frozen cube has melted.

8. A flavored cube for mixing a cocktail drink, comprising:
a packaging container;
a lid member detachably coupled to the packaging container; and
the flavored cube located inside the packaging container, the flavored cube having a liquid state and a frozen state.

9. The flavored cube of claim 8, wherein the flavored cube comprises cannabidiol (CBD).

10. The flavored cube of claim 8, wherein the flavored cube comprises tetrahydrocannabinol (THC).

11. The flavored cube of claim 8, wherein the packaging container defines an internal space substantially equal to 30 milliliters, and wherein in the liquid state the flavored cube defines a volume substantially equal to 25 milliliters, and in the frozen state the flavored cube defines a volume of less than 30 millimeters.

12. The flavored cube of claim 8, wherein the packaging container comprises plastic, wherein the lid member is adhesively attached to the packaging container, and wherein the flavored cube comprises at least three flavors.

13. The flavored cube of claim 12, wherein the at least three flavors define a flavor profile selected from the group consisting of: 1) a first flavor profile consisting of cucumber juice, watermelon puree and thyme; 2) a second flavor profile consisting of pink grapefruit juice, chili and tarragon; 3) a third flavor profile consisting of blood orange, simple syrup, cherry juice, bitters and ginger; 4) a fourth flavor profile consisting of Meyer lemon juice, bergamot, vanilla essence and chamomile; and 5) a fifth flavor profile consisting of peach nectar, Meyer lemon juice and Benedictine herbs.

14. A kit for mixing a cocktail drink, comprising:
a first packaging container and a second packaging container each defining an internal space to receive a first flavored cube and a second flavored cube, respectively;
the first flavored cube comprising a first plurality of ingredients, the first flavored cube having a liquid state and a frozen state;
the second flavored cube comprising a second plurality of ingredients, the second flavored cube having a liquid state and a frozen state; and
a first lid member and a second lid member that are each detachably coupled to the first packaging container and the second packaging container, respectively, wherein the first and second lids substantially seal the first and second packaging containers to thereby prevent the flavored cubes from spilling from the first and second packaging containers.

15. The kit of claim 14, wherein at least one of the first plurality of ingredients and the second plurality of ingredients comprises cannabidiol (CBD).

16. The kit of claim 14, wherein at least one of the first plurality of ingredients and the second plurality of ingredients comprises tetrahydrocannabinol (THC).

17. The kit of claim 14, further comprising a holding tray that receives the first and second packaging containers, wherein the holding tray is configured to receive at least 12 packaging containers.

18. The kit of claim 14, further comprising at least one of a martini shaker and a lowball glass.

19. The kit of claim 14, wherein at least one of the first plurality of ingredients and the second plurality of ingredients define a flavor profile selected from the group consisting of: 1) a first flavor profile consisting of cucumber juice, watermelon puree and thyme; 2) a second flavor profile consisting of pink grapefruit juice, chili and tarragon; 3) a third flavor profile consisting of blood orange, simple syrup, cherry juice, bitters and ginger; 4) a fourth flavor profile consisting of Meyer lemon juice, bergamot, vanilla essence and chamomile; and 5) a fifth flavor profile consisting of peach nectar, Meyer lemon juice and Benedictine herbs.

20. The kit of claim 14, wherein the internal space of the first and second packaging containers each defines a volume substantially equal to 30 milliliters.
